(19) United States
(12) Patent Application Publication
(10) Pub. No.: US 2012/0270303 A1
(43) Pub. Date: Oct. 25, 2012

(54) MANUFACTURING METHOD OF HIGH CONTENT OF STARCH FROM MICROALGAE

(75) Inventors: Chae Hwan Hong, Ansan (KR); Do Suck Han, Seongnam (KR)

(73) Assignee: HYUNDAI MOTOR COMPANY, Seoul (KR)

(21) Appl. No.: 13/155,830

(22) Filed: Jun. 8, 2011

(30) Foreign Application Priority Data
Apr. 20, 2011 (KR) 10-2011-0036868

(51) Int. Cl. C12N 1/12

(52) U.S. Cl. 435/257.1

(57) ABSTRACT

The present invention provides a medium for culturing microalgae comprising NaNO₃, K₂HPO₄, MgCl₂.6H₂O, Na₂CO₃, CaCl₂, ethylene diamine tetraacetate, citric acid and Na₂SiO₃.9H₂O or ferric citrate. The medium of the present invention can be used for culturing microalgae with high content of starch, and thus biomass materials can be obtained at a low production cost, leading to cost effectiveness. Consequently, the conventional petroleum-based polypropylene materials can be replaced with biomass-derived materials, which are applied to automotive interior and exterior materials. Therefore, considering the recent trend of high oil prices, dependence on petroleum-based products can be reduced, and production costs for interior and exterior materials can be also greatly reduced.

### CROSS-REFERENCE TO RELATED APPLICATION

This application claims under 35 U.S.C. §119(a) the benefit of Korean Patent Application No. 10-2011-0036868 filed Apr. 20, 2011, the entire contents of which are incorporated herein by reference.

### BACKGROUND

(a) Technical Field

The present invention relates to a medium for culturing microalgae with a high content of starch, and a method for culturing microalgae with a high content of starch using the same.

(b) Background Art

The 20^{th} century has brought about rapid industrialization which has attributed to the consumption of fossil fuels, in particular, petroleum, and the growing demand for petroleum has been driven by rapid industrialization and population growth. However, petroleum is not a renewable resource and has a limited reserve in the nature. In addition, carbon dioxide emissions from the burning of fossil fuels have been blamed for the main cause of global warming. With this regard, much attention is recently paid to energy-efficiency improvement and alternatives to petroleum in order to reduce carbon dioxide emissions.

One alternative to fossil fuels is a plant-derived, biomass polymer that is prepared from renewable plant resources such as corn, soybean, sugarcane, and wood by a chemical or biological method. This form of fuel has less of an environmental impact because it reduces carbon dioxide emissions rather than biodegradability. Among the biomass polymers, polylactic acid is a linear aliphatic polyester, and prepared by starch fermentation of corn and potato or by polymerization of sugar monomers that are obtained from glycosylation and fermentation of plant cellulose. It is also a carbon-neutral, environment-friendly, thermoplastic polymer resource. For large-scale production of biomaterials, however, it is important to secure an inexpensive biomass and sugar resources.

Of various biomass materials, increasing attention has been placed on microalgae, because microalgae are a promising sustainable source that grow quickly, have a high content of lipids, and do not compete with food resources. Of the biomass materials as alternative to fossil fuels, algae are considered to be a promising upcoming alternative that do not compete with food resources.

In particular, microalgae are a photosynthetic organism that is able to produce organic compounds from water and carbon dioxide by means of the solar energy. Generally, algae are largely classified into microalgae and macroalgae. Of many species of microalgae, microalgae with high content of lipids have been actively studied for the production of electric energy and biofuels.

As the biomass, microalgae are used in the production of biodiesel by transesterification, production of ethanol or methane by fermentation, production of methane or hydrogen by gasification, production of gas/liquid fuel by pyrolysis and production of heat or electric power by combustion. In fact, biodiesel production by transesterification of lipids contained in the biomass is fully commercialized worldwide. However, recovery of carbohydrates (pentose, hexose) through glycosylation requires a technique for culturing microalgae with high content of starch, which is still in its infancy in comparison.

Growth of algae generally demands silicon, a small amount of inorganic materials, vitamins or the like, in addition to essential elements such as nitrogen and phosphorus, and their growth range is determined by a proper gradient of each element. See Schindler 1974, Han, 2000, Lund, 1950. In nature, green algae actively grow at a high ratio of nitrogen/phosphorus, and small spherical cells such as Chlorella actively grow at a high ratio of nitrate or ammonia. Chlorella is a small unicellular phytoplankton and is a spherical cell with a diameter of about 3˜10 μm. It also grows rapidly under poor growth conditions including low light and temperature.

There have been about 30 different types of media for culturing Chlorella. Until now, the most widely known media for Chlorella is Allen's medium with high content of nitrogen and phosphorus. Such media are simply developed for the purpose of facilitating algae growth, but there have been no studies to develop media which can be used to maximize the content of starch in the microalgae.

The above information disclosed in this Background section is only for enhancement of understanding of the background of the invention and therefore it may contain information that does not form the prior art that is already known in this country to a person of ordinary skill in the art.

### SUMMARY OF THE DISCLOSURE

The present invention provides a specific medium for culturing microalgae to maximize the content of starch in the microalgae. In the illustrative embodiment, the present invention a medium for culturing microalgae includes NaNO₃, K₂HPO₄, MgCl₂.6H₂O, Na₂CO₃, CaCl₂, ethylene diamine tetraacetate, citric acid and Na₂SiO₃.9H₂O or ferric citrate. Additionally, the present invention also provides a method for culturing microalgae using the medium.

Other aspects and preferred embodiments of the invention are discussed infra.

### BRIEF DESCRIPTION OF THE DRAWINGS

The above and other features of the present invention will now be described in detail with reference to certain exemplary embodiments thereof illustrated the accompanying drawing which is given hereinbelow by way of illustration only, and thus are not limitative of the present invention, and wherein:

FIG. 1 is a photograph showing microalgae cultured using the medium of Example 1.

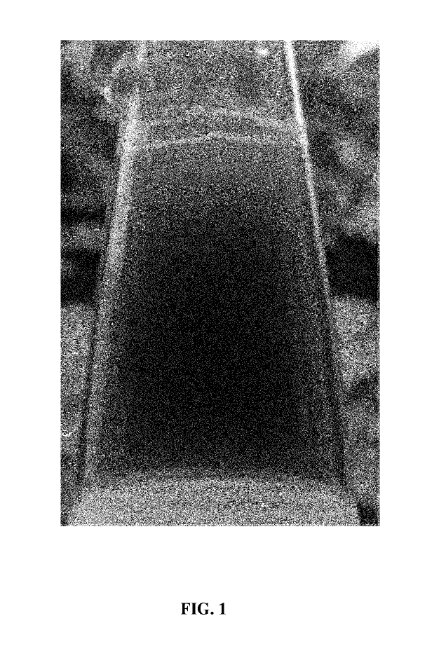

It should be understood that the appended drawings are not necessarily to scale, presenting a somewhat simplified representation of various preferred features illustrative of the basic principles of the invention. The specific design features of the present invention as disclosed herein, including, for example, specific dimensions, orientations, locations, and shapes will be determined in part by the particular intended application and use environment.

### DETAILED DESCRIPTION

Hereinafter reference will now be made in detail to various embodiments of the present invention, examples of which are illustrated in the accompanying drawings and described below. While the invention will be described in conjunction with exemplary embodiments, it will be understood that present description is not intended to limit the invention to those exemplary embodiments. On the contrary, the invention is intended to cover not only the exemplary embodiments, but also various alternatives, modifications, equivalents and other embodiments, which may be included within the spirit and scope of the invention as defined by the appended claims.

The present invention relates to a medium for culturing microalgae which includes about 80˜90 wt % of NaNO₃, about 2.0˜3.0 wt % of K₂HPO₄, about 4.0˜8.0 wt % of MgCl₂.6H₂O, about 1.0˜2.0 wt % of Na₂CO₃, about 1.0˜2.0 wt % of CaCl₂, about 1.0˜4.0 wt % of ethylene diamine tetraacetate, about 0.3˜0.5 wt % of citric acid and about 0.3˜0.6 wt % of Na₂SiO₃.9H₂O or ferric citrate.

The microalgae are preferably a microalga Chlorella, which is the most widely studied colony-type alga, and has a wide range of industrial uses. The cells undergo asexual reproduction and divide into 4, 8, or 16 cells, and grow in sea water or fresh water. There are a number of species well known to those skilled in the art. With difference between species, the growth and proliferation of Chlorella generally depends on temperature and light intensity provided during photosynthetic growth and proliferation. Thus, yield of Chlorella is greatly influenced by photoperiod and temperature. The concentration of organic nitrogen or carbon source is also an important factor together with temperature and pH control. The chemical composition of Chlorella greatly varies depending on conditions. The composition varies in a range of protein of about 50˜60%, sugar of about 15˜25%, and lipid of about 2˜65% depending on culture conditions. In particular, the content of lipid is highly variable.

In the present invention, a specific ingredient in the medium is controlled to regulate an increase in the number of individuals. Specifically, in order to prevent an increase in the number of individuals, the sulfur content in the medium is regulated. The present results showed that reduction of the sulfur content is very effective. As the sulfur content is reduced, cell division is remarkably reduced, and thus hydrocarbons within each cell are not utilized as an energy source, leading to an increase in starch content.

The medium for culturing microalgae has the following composition.

NaNO₃ (sodium nitrate) is the nitrogen source. Together with phosphorus, NaNO₃ is required for non-nitrogen-fixing algae as an essential nutrient, and is thus an essential element of the medium composition. The preferred content thereof is about 80˜90 wt %. Even when the nitrogen content is less than about 80 wt %, growth may not be affected. However, such nitrogen content is essential for enrichment culture. An excessive amount of nitrogen, on the other hand, may cause overgrowth of algae leading to deformation or necrosis.

K₂HPO₄ (dipotassium phosphate) is utilized as a source of phosphorus, and essential for synthesis of cytoplasmic proteins and nucleic acids, and thus it is an essential element constituting the medium composition, together with nitrogen. The preferred content thereof is about 2.0˜3.0 wt %, and it should not be lower than eutrophication parameters (25 relative to nitrogen). However, if the phosphorus content is too high, the growth may be inhibited, and in particular, the growth rate of undesired algae may exceed that of desired algae upon mixed cultivation.

MgCl₂.6H₂O is used herein, instead of MgSO₄.7H₂O which is generally used. It is a source of magnesium instead of MgSO₄.7H₂O, and also inhibits cell division while it does not affect synthesis of cellular proteins, amino acids, and chlorophyll because it is free of sulfate. That is, MgCl₂.6H₂O supplies magnesium, but does not supply sulfate, and thus hydrocarbons within each cell are not utilized as an energy source, leading to an increase in starch content. Notably, the preferred content thereof is about 4.0˜8.0 wt %.

Na₂CO₃ (sodium carbonate) plays an important role as well in the pumping action across the cell membrane, and acts as a source of carbon, and thus is an essential element constituting the medium composition. The preferred content is about 1.0˜2.0 wt %, and the excessive amount thereof may cause changes in hydrogen ion concentration, leading to necrosis.

CaCl₂ (calcium chloride) is a major source of calcium, and involved in metabolism through the cell membrane and membrane enzyme activities, and thus is an essential element constituting the medium composition. The preferred content is about 1.0˜2.0 wt %. If the content is too low, however, the efficiency of energy metabolism or membrane enzyme activities may be reduced. If the content is too high, it accumulates in the cell and causes cell aging.

Ethylene diamine tetraacetate (EDTA) binds with metal ions in the natural water or artificial water to supply them for cellular metabolism. However, an excessive amount thereof is harmful to cellular metabolism and cell wall, and deteriorates the effects of metal ions such as minerals. Therefore, the preferred content thereof is about 1.0˜4.0 wt %.

Citric acid is a conditional element as a carbon source in the medium composition, but essential for culturing green algae. The preferred content thereof is about 0.3˜0.5 wt %, and an excessive amount thereof, however, may cause a problem of changing the medium pH.

Na₂SiO₃.9H₂O (sodium metasilicate nonahydrate) is a source of silicon, and may be used in an amount of about 0.3˜0.6 wt %. If it is excluded, ferric citrate as a source of iron may be alternatively used in the same content of about 0.3˜0.6 wt %.

In addition to the above medium composition, one or more hydrates selected from the group consisting of H₃BO₃ (boric acid), MnCl₂.4H₂O (manganese chloride tetrahydrate), ZnSO₄.7H₂O (zinc sulfate heptahydrate), Na₂MoO₄.2H₂O (sodium molybdate dihydrate), CuSO₄.5H₂O (copper sulfate pentahydrate) and Co(NO₃)₂.6H₂O (cobalt nitrate hexahydrate) may be included, and the preferred content thereof is about 0.01˜0.5 wt %.

The present invention further relates to a method for culturing microalgae using the medium. Specifically, microalgae may be cultured under daytime conditions of about 33˜37° C. and about 770˜790 μmol/(m²S⁻¹) and night-time conditions of about 13˜17° C. and about 13˜17 μmol/(m²S⁻¹). Specifically, the microalgae may be a microalga Chlorella.

### EXAMPLES

Hereinafter, the present invention will be described in detail with reference to Examples. However, the scope of the invention is not intended to be limited by these Examples.

### Example

Chlorella vulgaris Beijerinck (Korean Marine Microalgae Culture Center, FC-015) was used. It was cultured at a daytime light intensity of 780 mmol/(m²S⁻¹) and at a night-time light intensity of 15 μmol/(m²S⁻¹) and the temperature was maintained at a daytime temperature of approximately 35° C. and at a night-time temperature of approximately 15° C.

The medium was prepared according to the following composition of Table 1.

TABLE 1

| | | | Com- | Com- | Com- |
|---|---|---|---|---|---|
| | | Ex- | parative | parative | parative |
| wt % | Example 1 | ample 2 | Example 1 | Example 2 | Example 3 |
| NaNO₃ | 86.8 | 87.3 | 86.8 | 87.3 | 85.3 |
| K₂HPO₄ | 2.0 | 2.0 | 2.0 | 2.0 | 2.0 |
| MgCl₂•6H₂O | 4.5 | 4 | — | — | — |
| MgSO₄•7H₂O | — | — | 4.5 | 4 | 6 |
| Na₂CO₃ | 1.3 | 1.3 | 1.3 | 1.3 | 1.3 |
| CaCl₂ | 1.8 | 1.8 | 1.8 | 1.8 | 1.8 |
| EDTA | 2.5 | 2.5 | 2.5 | 2.5 | 2.5 |
| Citric | 0.5 | 0.5 | 0.5 | 0.5 | 0.5 |
| acid | | | | | |
| Na₂SiO₃•9H₂O | 0.5 | 0.5 | 0.5 | 0.5 | 0.5 |
| H₃BO₃ | 0.1 | 0.1 | 0.1 | 0.1 | 0.1 |

### Test Examples

After cultivation for 30 hours according to the method of Example, dry weight and starch content were measured in the following manner. The results are shown in the following Table 2.

[Analysis Method]
Analysis of dry weight

About 5 cc of culture medium was taken, and washed with distilled water, followed by centrifugation. Subsequently, the resultant was dried at 100° C. for 12 hours, and then its weight was measured.

Measurement of Starch Content

About 5 cc of cultured microalgae was taken and centrifuged. Subsequently, microalgae were frozen at −20° C., and then finely pulverized. 80 vol % of ethanol was mixed therewith five times, and pigments were extracted. Then, 30 vol % of perchloric acid was added to the residue, and mixed, and centrifuged. The extraction was repeated until total 10 cc of the extract was obtained. 0.5 cc thereof was taken, and mixed with an anthrone solution (2 g of anthrone in 1 L of 72 vol % of sulfuric acid), and left in a 100° C. water bath for 10 min. The temperature was decreased to room temperature, and then absorbance was measured at 625 nm using a UV-VIS spectrophotometer. A calibration curve for glucose was generated.

TABLE 2

| | Example | | Comparative Example | | |
|---|---|---|---|---|---|
| Section | 1 | 2 | 1 | 2 | 3 |
| Dry weight | 1.4 g/L | 1.5 g/L | 3.1 g/L | 2.9 g/L | 3.0 g/L |
| Starch content in dry | 60 | 59 | 16 | 16 | 15 |
| weight (wt %) | | | | | |

As shown in the results, Comparative Examples showed about 16% of the starch content in dry weight (%) after 30 hours of cultivation, but Examples showed about 60 wt %, indicating that the total starch content within the final number of cells of Examples was twice or more than that of Comparative Examples.

### Effect of the Invention

The medium of the present invention can be used for culturing microalgae with high content of starch, and thus biomass materials can be prepared at a low production cost, thereby providing cost effective options. Consequently, the conventional petroleum-based polypropylene materials can be replaced with biomass-derived materials, which are applied to automotive interior and exterior materials. Therefore, considering the recent trend of high oil prices, dependence on petroleum-based products can be reduced, and production costs for interior and exterior materials can be also greatly reduced.

The invention has been described in detail with reference to preferred embodiments thereof. However, it will be appreciated by those skilled in the art that changes may be made in these embodiments without departing from the principles and spirit of the invention, the scope of which is defined in the appended claims and their equivalents.

What is claimed is:

1. A medium for culturing microalgae, comprising NaNO₃, K₂HPO₄, MgCl₂.6H₂O, Na₂CO₃, CaCl₂, ethylene diamine tetraacetate, citric acid and Na₂SiO₃.9H₂O or ferric citrate.

2. The medium of claim 1, wherein the medium comprises 80˜90 wt % of NaNO₃, 2.0˜3.0 wt % of K₂HPO₄, 4.0˜8.0 wt % of MgCl₂.6H₂O, 1.0˜2.0 wt % of Na₂CO₃, 1.0˜2.0 wt % of CaCl₂, 1.0˜4.0 wt % of ethylene diamine tetraacetate, 0.3˜0.5 wt % of citric acid and 0.3˜0.6 wt % of Na₂SiO₃.9H₂O or ferric citrate.

3. The medium of claim 1, wherein the medium further comprises 0.01˜0.5 wt % of one or more hydrates selected from the group consisting of H₃BO₃, MnCl₂.4H₂O, ZnSO₄.7H₂O, Na₂MoO₄.2H₂O, CuSO₄.5H₂O and Co(NO₃)₂.6H₂O.

4. A method for culturing microalgae using the medium; comprising:
culturing microalgae utilizing a medium containing NaNO₃, K₂HPO₄, MgCl₂.6H₂O, Na₂CO₃, CaCl₂, ethylene diamine tetraacetate, citric acid and Na₂SiO₃.9H₂O or ferric citrate.

5. The method of claim 4, further comprising culturing the microalgae under daytime conditions of 33˜37° C. and 770˜790 μmol/(m²S⁻¹) and night-time conditions of 13˜17° C. and 13˜17 μmol/(m²S⁻¹).

6. The method of claim 4, wherein the medium further comprises 80˜90 wt % of NaNO₃, 2.0˜3.0 wt % of K₂HPO₄, 4.0˜8.0 wt % of MgCl₂.6H₂O, 1.0˜2.0 wt % of Na₂CO₃, 1.0˜2.0 wt % of CaCl₂, 1.0˜4.0 wt % of ethylene diamine tetraacetate, 0.3˜0.5 wt % of citric acid and 0.3˜0.6 wt % of Na₂SiO₃.9H₂O or ferric citrate.

7. The method of claim 4, wherein the medium further comprises 0.01˜0.5 wt % of one or more hydrates selected from the group consisting of H₃BO₃, MnCl₂.4H₂O, ZnSO₄.7H₂O, Na₂MoO₄.2H₂O, CuSO₄.5H₂O and Co(NO₃)₂.6H₂O.
